(19) United States
(12) Patent Application Publication
(10) Pub. No.: US 2025/0318816 A1
(43) Pub. Date: Oct. 16, 2025

(54) SYSTEMS AND METHODS FOR ARRANGING CHANNELS OF AN ELONGATE FLEXIBLE DEVICE

(71) Applicant: Intuitive Surgical Operations, Inc., Sunnyvale, CA (US)

(72) Inventors: Samuel Selle, Sunnyvale, CA (US); Camille Calvin, San Jose, CA (US); Damini Ashok Chopra, San Jose, CA (US); Miles S. Joseph, Sunnyvale, CA (US)

(21) Appl. No.: 19/175,729

(22) Filed: Apr. 10, 2025

Related U.S. Application Data
(60) Provisional application No. 63/632,967, filed on Apr. 11, 2024.

(51) Int. Cl. A61B 17/00; A61B 17/04

(52) U.S. Cl. CPC A61B 17/00234; A61B 17/0469; A61B 34/30; A61B 34/71; A61B 2017/00283; A61B 2017/00318; A61B 2034/303

(57) ABSTRACT

A system comprises an elongate flexible device and a tool removably couplable to a distal end of the elongate flexible device. The elongate flexible device comprises a first working channel, a second working channel, and an imaging lumen configured to receive an imaging device. An operational space extends between a target anatomy and a distal end of the first working channel, a distal end of the second working channel, and a distal end of the imaging lumen. The tool comprises a first extension member configured to extend distally from the elongate flexible device, and a second extension member configured to extend distally from the elongate flexible device. When the tool is coupled to the elongate flexible device, the first extension member and the second extension member are positioned outside of the operational space such that the operational space is free from obstruction by the first and second extension members.

### CROSS-REFERENCED APPLICATIONS

This application claims priority to and the benefit of U.S. Provisional Application No. 63/632,967 filed Apr. 11, 2024, and entitled “Systems and Methods for Arranging Channels of an Elongate Flexible Device,” which is incorporated by reference herein in its entirety.

### FIELD

Examples described herein relate to systems and methods for arranging channels of an elongate flexible device. More particularly, examples relate to systems and methods for arranging channels and lumens of the elongate flexible device to minimize obstruction of the channels and lumens when a tool or other instrument is coupled to a distal end of the elongate flexible device.

### BACKGROUND

Minimally invasive medical techniques may generally be intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions an operator may insert minimally invasive medical instruments such as therapeutic instruments, diagnostic instruments, imaging instruments, and surgical instruments. Some minimally invasive medical instruments may involve the use of endoscopic devices that are navigated to a target location to perform a procedure. Some minimally invasive medical instruments include more than one working channel and/or lumen. Systems and methods are needed to arrange the channels and lumens to minimize obstruction of the channels and lumens when a tool or other instrument is coupled to a distal end of the minimally invasive medical instruments.

### SUMMARY

The following presents a simplified summary of various examples described herein and is not intended to identify key or critical elements or to delineate the scope of the claims.

Consistent with some examples, a system is provided. The system includes an elongate flexible device including a first working channel including a distal end, a second working channel including a distal end, and an imaging lumen including a distal end. The imaging lumen is configured to receive an imaging device. An operational space extends between a target anatomy and the distal end of the first working channel, the distal end of the second working channel, and the distal end of the imaging lumen. The system further includes a tool removably couplable to a distal end of the elongate flexible device. The tool includes a first extension member configured to extend distally from the distal end of the elongate flexible device, and a second extension member configured to extend distally from the distal end of the elongate flexible device. When the tool is coupled to the distal end of the elongate flexible device, the first extension member and the second extension member are positioned outside of the operational space such that the operational space is free from obstruction by the first extension member and the second extension member.

Consistent with other examples, a system is provided. The system includes an elongate flexible device including a first working channel including a distal end, a second working channel including a distal end, and an imaging lumen including a distal end. The imaging lumen is configured to receive an imaging device. An operational space extends between a target anatomy and the distal end of the first working channel, the distal end of the second working channel, and the distal end of the imaging lumen. When a tool is coupled to a distal end of the elongate flexible device, a first extension member of the tool and a second extension member of the tool are positioned outside of the operational space such that the operational space is free from obstruction by the first extension member and the second extension member. The first extension member is configured to extend distally from the distal end of the elongate flexible device, and the second extension member configured to extend distally from the distal end of the elongate flexible device.

Consistent with other examples, a system is provided. The system includes an elongate flexible device including an imaging lumen including a distal end. The imaging lumen is configured to receive an imaging device. The elongate flexible device further includes an illumination lumen including a distal end. The illumination lumen is configured to receive an illumination device. An operational space extends between a target anatomy and the distal end of the imaging lumen and the distal end of the illumination lumen. When a tool is coupled to a distal end of the elongate flexible device illumination light output by the illumination device reflects off an extension member of the tool and creates a glare in an image captured by the imaging device. To reduce the glare in the image the extension member of the tool is positioned outside of the operational space such that the operational space is free from obstruction by the extension member. The illumination lumen is spaced from the extension member.

Consistent with other examples, a method is provided. The method includes receiving an image from an imaging device positioned within an imaging lumen of an elongate flexible device. The method further includes receiving illumination light output by an illumination device positioned within an illumination lumen of the elongate flexible device. The illumination light reflects off a first extension member of a tool coupled to the elongate flexible device, and the reflected illumination light creates a glare in the image captured by the imaging device. An operational space extends between a target anatomy and a distal end of the imaging lumen and a distal end of the illumination lumen. The first extension member of the tool is positioned outside of the operational space such that the operational space is free from obstruction by the first extension member to reduce the glare in the image. The illumination lumen is spaced from the first extension member to reduce the glare in the image.

Consistent with other examples, a system is provided. The system includes an elongate flexible device including an imaging lumen including a distal end. The imaging lumen is configured to receive an imaging device. The elongate flexible device further includes a fluid lumen including a distal end. A nozzle is positioned at a distal end of the fluid lumen. An operational space extends between a target anatomy and the distal end of the imaging lumen and the distal end of the fluid lumen. When a tool is coupled to a distal end of the elongate flexible device, a first extension member of the tool is positioned outside of the operational space such that the operational space is free from obstruction by the first extension member. The first extension member is configured to extend distally from the distal end of the elongate flexible device. The nozzle is aligned with the imaging lumen and the first extension member. When fluid is discharged from the distal end of the fluid lumen, the nozzle directs the fluid over the imaging device to clean the imaging device and over the first extension member to clean the first extension member.

Other examples include corresponding computer systems, apparatus, and computer programs recorded on one or more computer storage devices, each configured to perform the actions of any one or more methods described below.

It is to be understood that both the foregoing general description and the following detailed description are illustrative and explanatory in nature and are intended to provide an understanding of the various examples described herein without limiting the scope of the various examples described herein. In that regard, additional aspects, features, and advantages of the various examples described herein will be apparent to one skilled in the art from the following detailed description.

### BRIEF DESCRIPTIONS OF THE DRAWINGS

FIG. 1 is a simplified diagram of a patient anatomy according to some examples.

FIG. 2A illustrates a cross-sectional side view of an elongate flexible device with an operational space extending between a distal end of the elongate flexible device and a target anatomy according to some examples.

FIG. 2B illustrates a front view of the distal end of the elongate flexible device of FIG. 2A according to some examples.

FIG. 3A illustrates a cross-sectional side view of a tool that is removably couplable to a distal end of an elongate flexible device according to some examples.

FIG. 3B illustrates a front view of the tool of FIG. 3A according to some examples.

FIG. 4A illustrates a cross-sectional side view of a tool coupled to a distal end of an elongate flexible device according to some examples.

FIG. 4B illustrates a front view of the tool of FIG. 4A coupled to a distal end of an elongate flexible device according to some examples.

FIG. 5A illustrates a cross-sectional side view of a tool with an extension member including an optional attachment portion according to some examples.

FIG. 5B illustrates a front view of the tool of FIG. 5A according to some examples.

FIG. 6A illustrates a cross-sectional side view of a tool attached to a distal end of an elongate flexible device according to some examples.

FIG. 6B illustrates a front view of the tool of FIG. 6A coupled to the distal end of the elongate flexible device of FIG. 6A according to some examples.

FIG. 7 illustrates a front view of a distal end of an elongate flexible device according to some examples.

FIG. 8 illustrates a front view of a distal end of an elongate flexible device with pull wires according to some examples.

FIG. 9 illustrates a front view of a distal end of an elongate flexible device according to some examples.

FIG. 10 illustrates a front view of a distal end of an elongate flexible device with an imaging lumen and an illumination lumen according to some examples.

FIG. 11 illustrates a front view of a distal end of an elongate flexible device with a fluid lumen and an imaging lumen according to some examples.

FIG. 12A illustrates a cross-sectional side view of a suturing tool coupled to a distal end of an elongate flexible device according to some examples.

FIG. 12B illustrates a front view of the suturing tool of FIG. 12A coupled to a distal end of an elongate flexible device according to some examples.

FIG. 13 illustrates a front view of a suturing tool coupled to a distal end of an elongate flexible device according to some examples.

FIG. 14 is a flowchart illustrating a method of visualizing a target anatomy based on images received from an imaging device of an elongate flexible device according to some examples.

FIG. 15 is a schematic diagram for a robotically-assisted manipulator system according to some examples.

FIG. 16A is a schematic diagram of an instrument system according to examples.

FIG. 16B illustrates a distal portion of the instrument system of FIG. 16A with an extended example of an instrument according to some examples.

Various examples described herein and their advantages are described in the detailed description that follows. It should be appreciated that like reference numerals are used to identify like elements illustrated in one or more of the figures for purposes of illustrating but not limiting the various examples described herein.

### DETAILED DESCRIPTION

In the following description, specific details describe some examples consistent with the present disclosure. Numerous specific details are set forth in order to provide a thorough understanding of the examples. It will be apparent to one skilled in the art, however, that some examples may be practiced without some or all of these specific details. The specific examples disclosed herein are meant to be illustrative but not limiting. One skilled in the art may realize other elements that, although not specifically described, are within the scope and the spirit of this disclosure. In addition, to avoid unnecessary repetition, one or more features shown and described in association with one example may be incorporated into other examples unless specifically described otherwise or if the one or more features would make an example non-functional. In some instances, well known methods, procedures, components, and circuits have not been described in detail so as not to unnecessarily obscure aspects of the examples.

Aspects of this disclosure herein can be part of a computer-assisted teleoperational manipulator system, sometimes referred to as a robotically-assisted manipulator system or a robotic system. The manipulator system can include one or more manipulators that can be operated with the assistance of an electronic controller (e.g., computer) to move and control functions of one or more instruments when coupled to the manipulators.

In some examples, an elongate flexible device (e.g., an endoscope) includes multiple working channels and/or lumens. The arrangement and positions of the working channels and lumens may be adjusted to minimize obstruction of the working channels and lumens when a tool (e.g., a suturing tool, a diagnostic tool, a therapeutic tool) or other instrument is coupled to a distal end of the elongate flexible device. The flexibility in arranging the working channels and lumens may help to: reduce glare caused by an illumination device in an image captured by an imaging device, more accurately determine a shape of the elongate flexible device, clean multiple components with a single discharge of fluid, and obtain a maximum bite depth for a needle when the tool is a suturing tool.

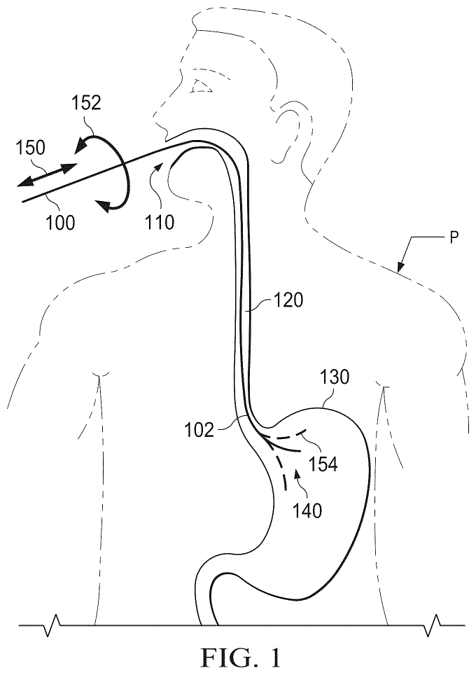

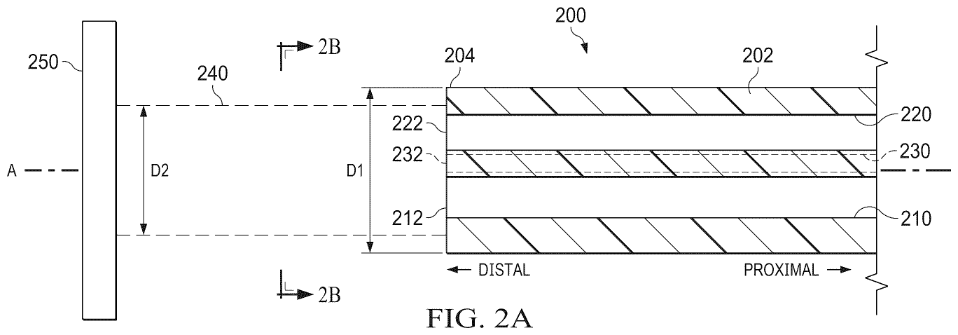

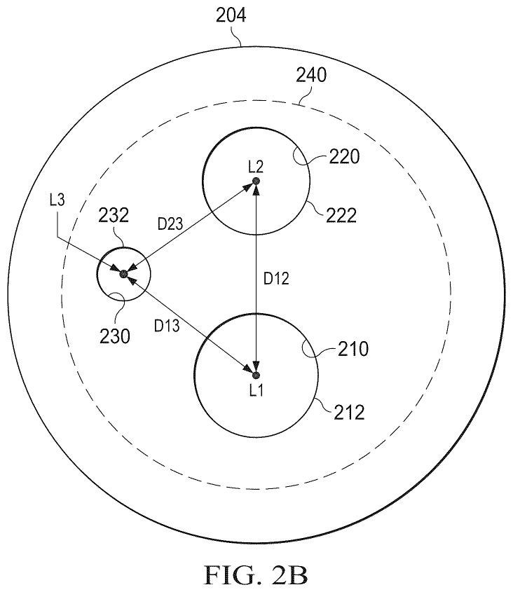

FIG. 1 illustrates an elongate flexible device 100 extending through an anatomic orifice 110 such as a mouth of a patient P, within an anatomic passageway 120 such as the esophagus of the patient P, and into an anatomic structure 130. In various examples, the elongate flexible device 100 may be a flexible catheter or endoscope (e.g., gastroscope, bronchoscope) and may optionally include one or more working channels sized and shaped to receive one or more medical instruments (see, e.g., FIGS. 2A-2B). In some examples, the anatomic structure 130 may be a stomach. The anatomy of the patient P may have an anatomical frame of reference (XA, YA, ZA). A distal portion 102 of the elongate flexible device 100 may be used to perform a medical procedure, such as a suturing, biopsy, and/or ablation procedure, at or near a target location 140 located in the anatomic structure 130 using any of the methods or systems described herein. In some examples, the target location 140 may be in the gastrointestinal tract, such as in the stomach, in the intestines, at or near a gastroesophogeal junction or the pylorus, or at other locations along the gastrointestinal tract. The elongate flexible device 100 may be advanced or retracted in a longitudinal degree of freedom of motion 150 and/or rotate in a rotational or roll degree of freedom of motion 152 (e.g., roll relative to a longitudinal axis of the elongate flexible device 100). The distal portion 102 of the elongate flexible device 100 may also be articulatable in steering degrees of freedom of motion 154 (e.g., pitch and/or yaw orientations).

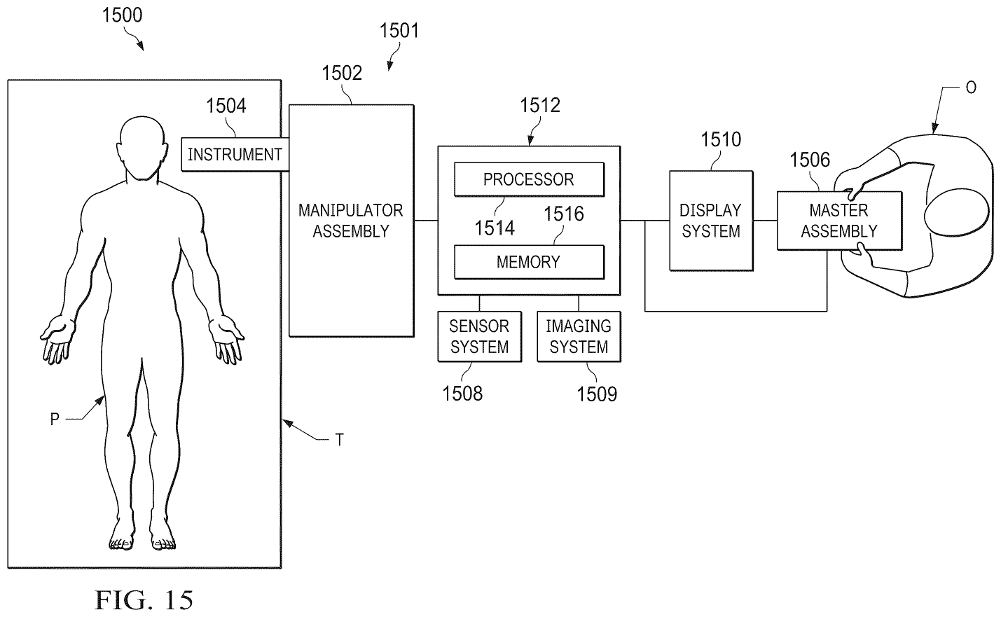

In some examples, the elongate flexible device 100 may be initially inserted and navigated to the target location 140 using full manual control. Full manual control involves one or more operators employing hand manipulations and forces to the elongate flexible device 100 to control longitudinal motion 150 (insertion/retraction) and roll motion 152 of the elongate flexible device 100. For example, the operator may apply manual forces to insert and/or retract the elongate flexible device 100 and/or to rotate the elongate flexible device 100 about its longitudinal axis. In some examples, the elongate flexible device 100 may be connected to a robotically-assisted manipulator assembly (e.g., manipulator assembly 1502 of FIG. 15) but may be operated in a full manual control mode of the robotically-assisted medical system that allows an operator to manually manipulate the elongate flexible device with full manual control. In some examples, the elongate flexible device 100 may be operated with robotically-assisted control modes. For example, robotically-assisted control modes may be activated to control one or more of longitudinal motion 150 (insertion/retraction), roll motion 152, or steering motion 154 of the elongate flexible device 100.

FIG. 2A illustrates a cross-sectional side view of an elongate flexible device 200 with an operational space 240 extending between a distal end 204 of the elongate flexible device 200 and a target anatomy 250. The elongate flexible device 200 includes a body portion 202 and the distal end 204. In some examples, the elongate flexible device 200 is the elongate flexible device 100 of FIG. 1. Thus, in various examples, the elongate flexible device 200 may be a flexible catheter or endoscope (e.g., gastroscope, bronchoscope) and may optionally include one or more working channels and/or lumens sized and shaped to receive one or more medical instruments, tools, or devices. For example, the elongate flexible device 200 includes a working channel 210 (e.g., a lumen), a working channel 220 (e.g., a lumen), and an imaging lumen 230. In some examples, a diameter of the working channel 210 is larger than a diameter of the working channel 220. Alternatively, the diameter of the working channel 220 may be larger than or the same as the diameter of the working channel 210. The working channel 210 may be a main working channel, and the working channel 220 may be a secondary working channel. Although in some examples, the working channel 220 is the main working channel, and the working channel 210 is the secondary working channel.

The working channel 210 and the working channel 220 are sized and shaped to receive one or more medical instruments that may be inserted into and through the elongate flexible device 200. The medical instruments may include one or more of a biopsy instrument, an ablation instrument, an imaging instrument, a stapler, a cauterizing instrument, a suturing instrument, a grasper, a clamp, or any other medical instrument used to interact with and/or manipulate anatomy of the patient, such as the target anatomy 250 for example. The imaging lumen 230 is sized and shaped to receive an imaging instrument, such as a camera, which may be a stereoscopic camera or other type of camera.

The operational space 240 defines a working area in which the medical instruments that extend out of the working channel 210, the working channel 220, and/or the imaging lumen 230 may move and be manipulated. The working area defined by the operational space 240 allows room for the medical instruments to manipulate, observe, or otherwise interact with the target anatomy 250. The target anatomy 250 may be any type of lesion, tumor, tissue, or other anatomy of interest, such as a suture site, treatment site, or biopsy site. The operational space 240 extends between the target anatomy 250 and a distal end 212 of the working channel 210, a distal end 222 of the working channel 220, and a distal end 232 of the imaging lumen 230. In some examples, a diameter D2 of the operational space 240 is smaller than a diameter D1 of the distal end 204 of the elongate flexible device 200. In other examples, the diameter D2 of the operational space 240 is the same as the diameter D1 of the distal end 204 of the elongate flexible device 200. The operational space 240 extends in an axial direction along a longitudinal axis A of the elongate flexible device 200.

FIG. 2B illustrates a front view of the distal end 204 of the elongate flexible device 200 looking in the proximal direction from the target anatomy 250. The working channel 210 includes a longitudinal axis L1, the working channel 220 includes a longitudinal axis L2, and the imaging lumen 230 includes a longitudinal axis L3. The longitudinal axis L1 of the working channel 210 is spaced from the longitudinal axis L2 of the working channel 220 by a distance D12. The longitudinal axis L1 of the working channel 210 is spaced from the longitudinal axis L3 of the imaging lumen 230 by a distance D13. The longitudinal axis L2 of the working channel 220 is spaced from the longitudinal axis L3 of the imaging lumen 230 by a distance D23. In some examples, the working channel 210, the working channel 220, and the imaging lumen 230 are equidistant from each other such that the distances D12, D13, and D23 are all the same length. In other examples, the distances, D12, D13, and D23 may each be different lengths. In still other examples, the distances D12 and D13 may be the same length, but the distance D23 may be shorter than the distances D12 and D13. Any other combination of varying lengths for the distances D12, D13, and D23 may be used depending on the configuration of the elongate flexible device 200. For example, the working channel 210, the working channel 220, and the imaging lumen 230 may be arranged in the elongate flexible device 200 at any position within the body portion 202 of the elongate flexible device 200. The working channels and lumens may be arranged according to the type of elongate flexible device 200 being used or according to the types of medical instruments positioned within the working channels and lumens. Different arrangements and spacing between the working channels and lumens may allow for different medical procedures to be performed and for different combinations of instruments to be used during the medical procedures. For all of the variations of spacings and arrangements of the working channels and lumens, the distal ends of the working channels and lumens may remain within the operational space 240. In this regard, the shape of the operational space 240 may change based on the arrangement of the working channels and lumens such that operational space 240 continues to surround the distal ends of the working channels and lumens. In other examples, some of the working channels and lumens may be outside of the operational space 240, as discussed in greater detail below.

When determining what arrangement of the working channels and lumens of the elongate flexible device 200 should be used for a particular procedure, some design features to consider are a distance between the longitudinal axis L1 of the working channel 210 and the longitudinal axis of the elongate flexible device 200, the outer diameter of the working channel 210, the diameter of the working channel 220, and the diameter of the distal end 204 of the elongate flexible device 200. Other design features may also be considered depending on the particular medical procedure to be performed using the elongate flexible device 200.

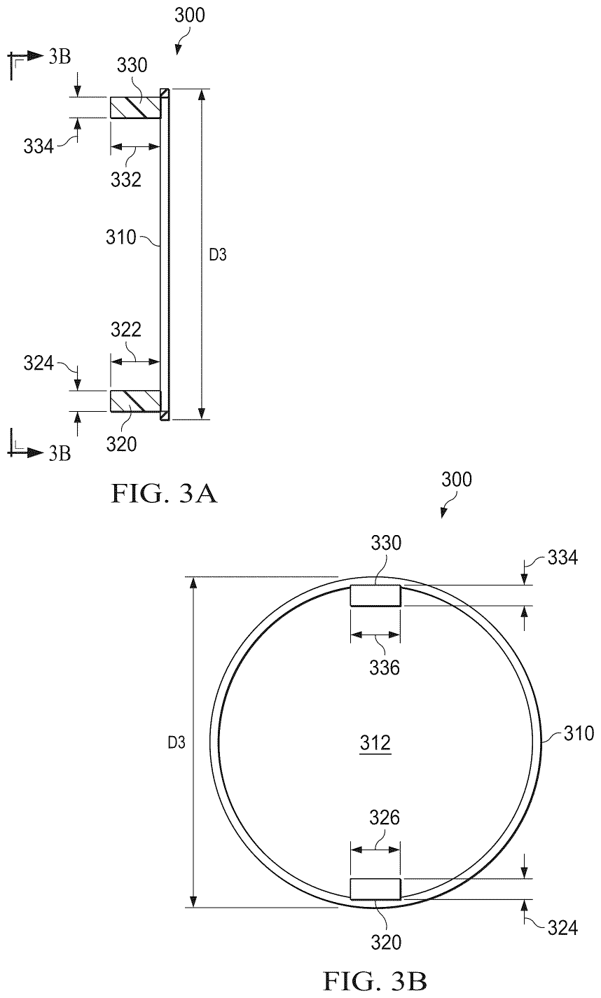

FIG. 3A illustrates a cross-sectional side view of a tool 300. The tool 300 is removably couplable to the distal end 204 of the elongate flexible device 200. Generally, the tool 300 can be a device that has components that are too large to insert via a working channel, although some components of the tool 300 can operate within the working channel, such as actuation or electrical cables. Accordingly, the tool 300 or a distal portion thereof can be configured to removably couple to the distal end 204 of the elongate flexible device 200. Examples of the tool 300 can include retrieval devices, electrosurgical devices, viewing attachments, stapling devices, and suturing devices. Because of the relatively large size of the tool 300 as compared to the distal end 204, all or portions of the tool 300 can block camera views and/or working channels of prior endoscopes. The tool 300 includes a rim 310, an extension member 320 (which may be a first extension member), and an extension member 330 (which may be a second extension member). FIG. 3B illustrates a front view of the tool 300. The rim 310 includes an outer diameter D3 and defines an interior opening 312. In some examples, the extension member 320 and the extension member 330 are coupled to the rim 310. The extension member 320 includes a length 322, a height 324, and a width 326. The length 322 is shown as being greater than the height 324, and the width 326 is shown as being greater than the height 324. However, any other dimensions may be used for the length 322, the height 324, and the width 326 of the extension member 320. In some examples, some or all of the length 322, the height 324, and the width 326 are the same length. The extension member 330 includes a length 332, a height 334, and a width 336. The length 332 is shown as being greater than the height 334, and the width 336 is shown as being greater than the height 334. However, any other dimensions may be used for the length 332, the height 334, and the width 336 of the extension member 330. In some examples, some or all of the length 332, the height 334, and the width 336 are the same length. Various types of extension members may be used depending on the type of tool that is used. These various types of extension members may have different dimensions. In some examples, the extension members 320 and 330 may be non-rectangular in shape and therefore may have different dimensions other than a length, width, and height.

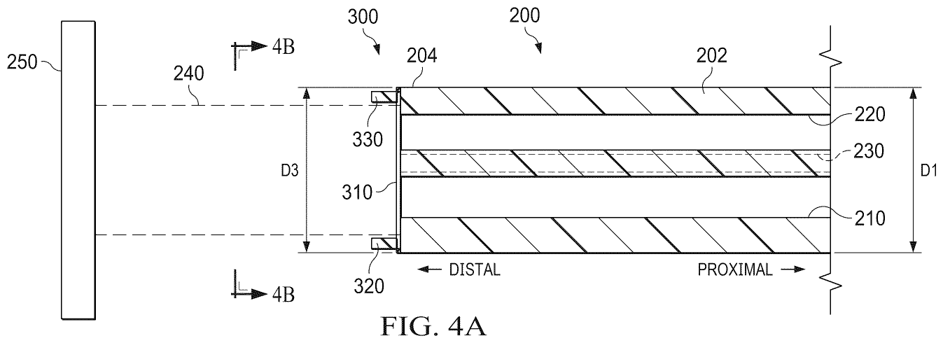

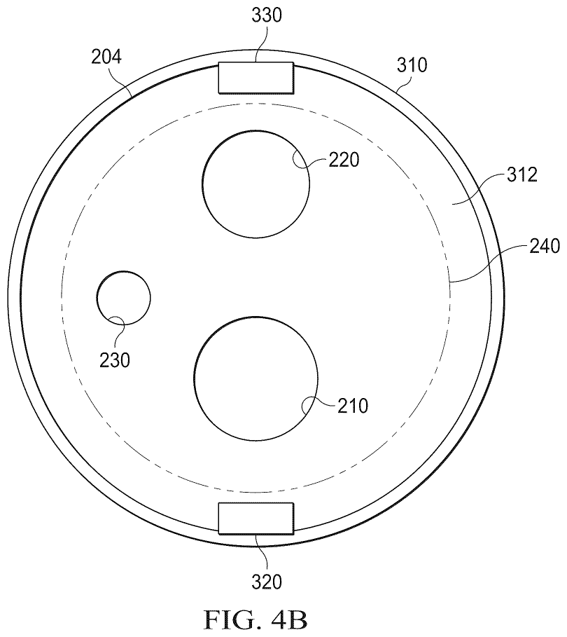

FIG. 4A illustrates a cross-sectional side view of the tool 300 coupled to the distal end 204 of the elongate flexible device 200. FIG. 4B illustrates a front view of the tool 300 coupled to the distal end 104 of the elongate flexible device 200. In some examples, the outer diameter D3 of the rim 310 of the tool 300 is larger than the diameter D1 of the distal end 204 of the elongate flexible device 200. This allows the rim 310 of the tool 300 to fit around the distal end 204 of the elongate flexible device 200. In such examples, the tool 300 may be coupled to the distal end 204 via a press fit connection, a threaded connection, a bonded connection (e.g., an adhesive connection, a welded connection), or via any other mechanical connection. In other examples, the outer diameter D3 of the rim 310 is the same as or smaller than the diameter D1 of the distal end 204. In such examples, the tool 300 may be coupled to the distal end 204 via a bonded connection (e.g., an adhesive connection, a welded connection) or via any other suitable mechanical connections.

FIGS. 4A and 4B show that the extension members 320, 330 are positioned outside of the operational space 240. Thus, the extension members 320, 330 do not interfere with the medical instruments that are positioned within and/or extend out from the working channel 210, the working channel 220, and the imaging lumen 230. For example, by positioning the extension members 320, 330 outside of the operational space 240, the medical instruments may be able to operate with their full range of motion available in the operational space 240. Additionally, when the extension members 320, 330 are positioned outside of the operational space 240, the extension members 320, 330 do not obstruct the view of the imaging device positioned within the imaging lumen 230. This allows for a full view of the operational space 240 to be captured, which may allow for a full view of the surgical procedure at the target anatomy 250 to be imaged and recorded by the imaging device.

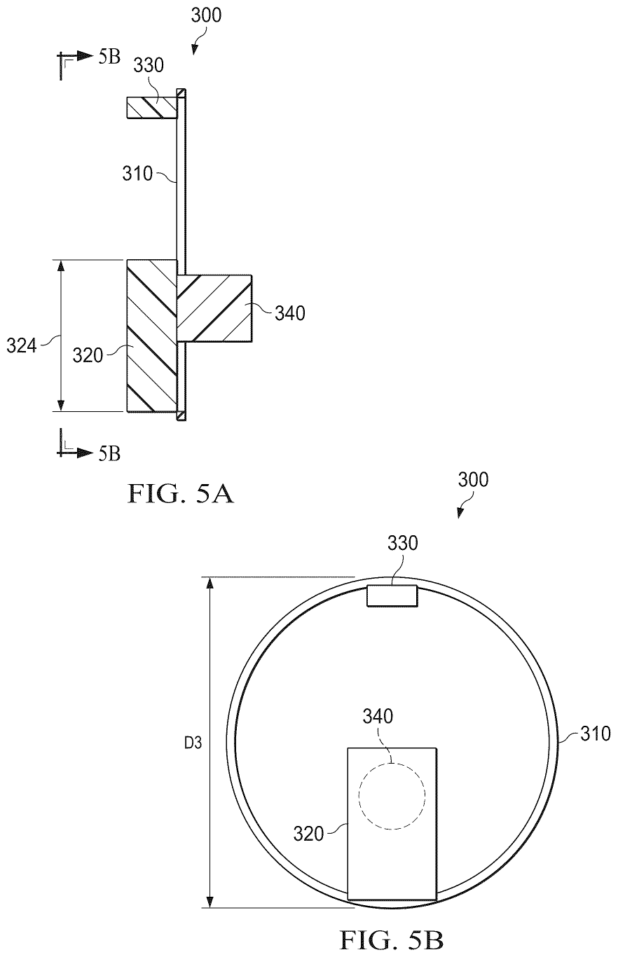

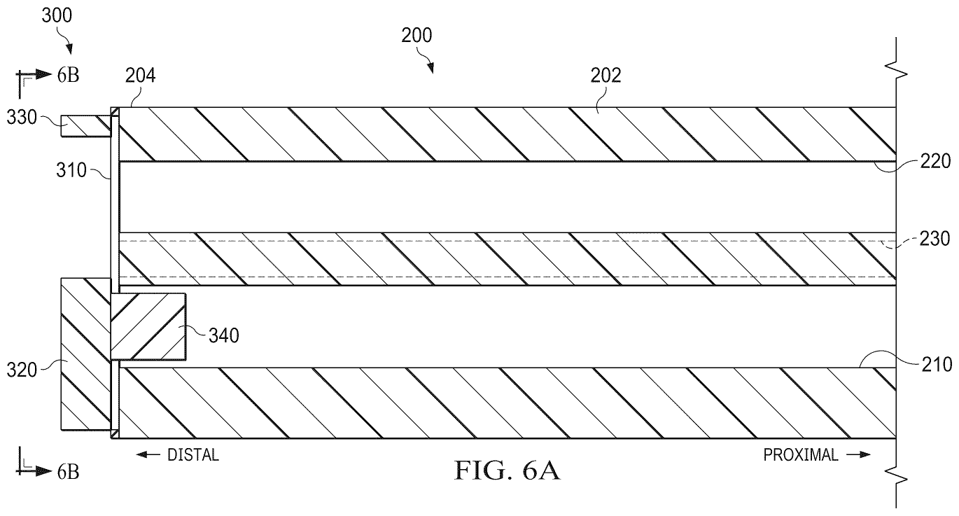

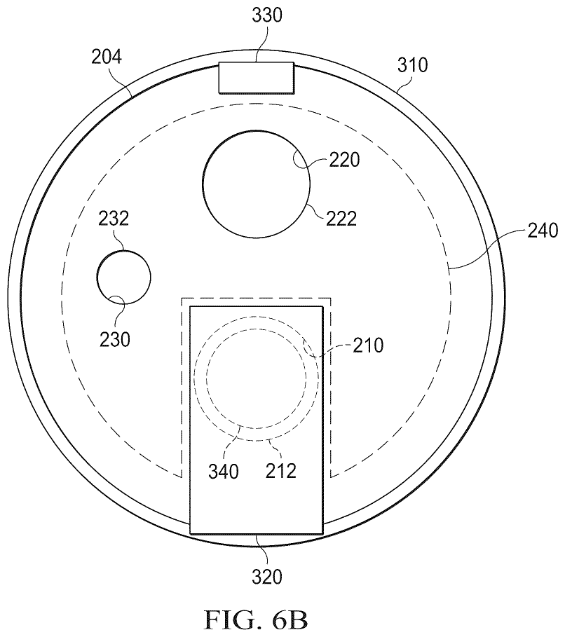

FIG. 5A illustrates a cross-sectional side view of the tool 300 with the extension member 320 including an optional attachment portion 340. The attachment portion 340 extends in a proximal direction. In some examples, the attachment portion 340 is used to assist with coupling the tool 300 to the distal end 204 of the elongate flexible device 200. FIG. 5B illustrates a front view of the tool 300 with the extension member 320 including the attachment portion 340. FIG. 6A illustrates a cross-sectional side view of the tool 300 attached to the distal end 204 of the elongate flexible device 200. FIG. 6B illustrates a front view of the tool 300 coupled to the distal end 204 of the elongate flexible device 200. When the tool 300 is coupled to the distal end 204, the attachment portion 340 of the extension member 320 extends into the working channel 210. The attachment portion 340 includes a locking feature (not shown) that rotationally locks the tool 300 relative to the elongate flexible device 200. The locking feature may also axially lock the tool 300 relative to the elongate flexible device 200. The locking feature may include one or more of a balloon, an expandable membrane, expandable springs, or other expandable features that may be deployed when the attachment portion 340 is within the working channel 210.

In some examples, to ensure that the tool 300 is coupled to the elongate flexible device 200 in the desired position and orientation, alignment markers and/or guidance tools may be used. For example, the working channel 210 may act as a guidance tool. When the attachment portion 340 of the extension member 320 is received within the working channel 210, the working channel 210 causes the tool 300 to be axially aligned with the elongate flexible device 200. The tool 300 may be axially aligned with the elongate flexible device 200 when a longitudinal axis of the tool 300 is colinear with the longitudinal axis of the elongate flexible device 200. The attachment portion 340 of the extension member 320 may also include a locking feature (e.g., a spring or other expandable member) that rotationally locks the tool 300 with respect to the elongate flexible device 200. Thus, the tool 300 may be axially and rotationally locked with respect to the elongate flexible device 200 in some examples. Other alignment markers may be positioned on the distal end 204 of the elongate flexible device 200 to help couple the tool 300 to the elongate flexible device 200 in the desired position and orientation.

As shown in FIG. 6B, when the attachment portion 340 extends into the working channel 210, the extension member 320 covers the distal end 212 of the working channel 210. Therefore, the boundary of the operational space 240 shifts. As shown in FIG. 6B, the operational space 240 extends from the distal end 222 of the working channel 220 and from the distal end 232 of the imaging lumen 230. The operational space 240 does not extend from the distal end 212 of the working channel 210 because the extension member 320 covers the working channel 210 and because the attachment portion 340 of the extension member 320 extends into the working channel 210. The boundary of the operational space 240 can shift in other ways depending on the positions and sizes of the extension member 320 and the extension member 330.

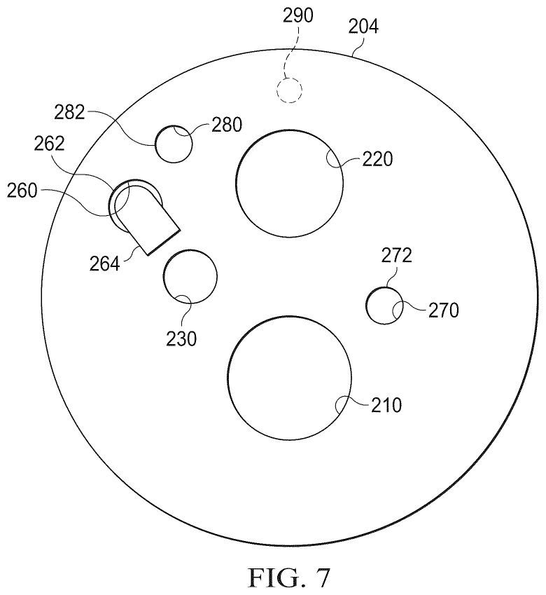

FIG. 7 illustrates a front view of the distal end 204 of the elongate flexible device 200 with additional optional features. For example, the elongate flexible device 200 optionally also includes one or more of a fluid lumen 260, an irrigation lumen 270, an illumination lumen 280, and a sensor lumen 290. As discussed above, in some examples, each of the working channels and lumens of the elongate flexible device 200 may be positioned within the operational space 240. In some alternative examples, some of the working channels and/or lumens of the elongate flexible device 200 are obstructed by the tool 300 when the tool 300 is coupled to the elongate flexible device 200. For example, one or more of the working channel 220, the imaging lumen 230, the fluid lumen 260, the irrigation lumen 270, and the illumination lumen 280 may be fully or partially obstructed by the extension member 320 and/or the extension member 330 of the tool 300 when the tool 300 is coupled to the elongate flexible device 200. Therefore, one or more of the working channels and lumens may be fully or partially positioned outside of the operational space 240.

In some examples, the working channel 210, the working channel 220, and the imaging lumen 230 may be positioned as close to the longitudinal axis of the elongate flexible device 200 as possible. This may provide more available space in the elongate flexible device 200 to place additional working channels or lumens. In some examples, the working channel 210, the working channel 220, and the imaging lumen 230 may be positioned as close together as possible. This may allow for the imaging device in the imaging lumen 230 to have more visibility of the medical instruments or tools that extend out of working channel 210 and/or the working channel 220 than if the imaging lumen 230 was positioned farther away from the working channel 210 and/or the working channel 220. In some examples, the imaging lumen 230 may be positioned as close to the longitudinal axis of the elongate flexible device 200 as possible. This may allow for the imaging device in the imaging lumen 230 to be able to view more of the components that may be positioned in the operational space 240. This may also reduce the likelihood that the imaging device is obstructed or partially obstructed by any other component extending through the elongate flexible device 200 or coupled to the tool 300.

Fluid may be provided to the distal end 204 of the elongate flexible device 200 and sometimes into the operational space 240 through the fluid lumen 260. The fluid may be one or more of a liquid (e.g., saline) or a gas (e.g., air). In some examples, insufflation fluid may be provided through the fluid lumen 260. A nozzle 264 may be located at a distal end 262 of the fluid lumen 260. The nozzle 264 is used to direct the fluid from the fluid lumen 260 and onto the imaging device that is positioned within the imaging lumen 230. This can clean a lens of the imaging device to help the imaging device capture clear pictures of the target anatomy 250 and the medical instrument(s) that may be positioned in the operational space.

Irrigation fluid may be provided to the distal end 204 of the elongate flexible device 200 and into the operational space 240 through the irrigation lumen 270. The irrigation fluid may be used to wash or remove matter (e.g., blood, mucus) from the target anatomy 250 or from locations near the target anatomy 250. Additionally or alternatively, the irrigation fluid may be used to wash or remove matter from one or more of the extension member 320, the extension member 330, the rim 310, or any one or more of the medical instruments that may be extended from or positioned within the working channel 220 or the lumens 230, 260, 270, 280 discussed above. To remove the irrigation fluid and/or the removed matter, the working channel 220 is optionally used to provide suction.

Illumination light may be provided to the distal end 204 of the elongate flexible device 200 and into the operational space 240 through the illumination lumen 280. In some examples, an illumination device, such as an optical fiber, may be positioned within the illumination lumen 280. The illumination device may be any other type of illumination device. The illumination device may extend to or past a distal end 282 of the illumination lumen 280. The illumination device may illuminate the target anatomy 250 and any surrounding areas. In some examples, the elongate flexible device 200 includes more than one illumination lumen 280. A separate illumination device may be positioned within each, respective illumination lumen 280 such that multiple illumination devices are used to provide illumination from the distal end 204 of the elongate flexible device 200.

In some examples, the illumination light reflects off of the extension member 320 or the extension member 330. In some examples, the illumination light reflects off of both the extension member 320 and the extension member 330. The reflection of the illumination light off of the extension member 320 and/or the extension member 330 may create a glare in the image captured by the imaging device.

To reduce the glare, the extension member 320 and/or the extension member 330 may be positioned outside of the operational space 240. Additionally or alternatively, the illumination lumen 280 may be spaced from the extension member 320 and/or the extension member 330 to reduce the glare in the image captured by the imaging device. Additionally or alternatively, the extension member 320 and/or the extension member 330 may be coated with a glare reduction coating to reduce the glare in the image captured by the imaging device.

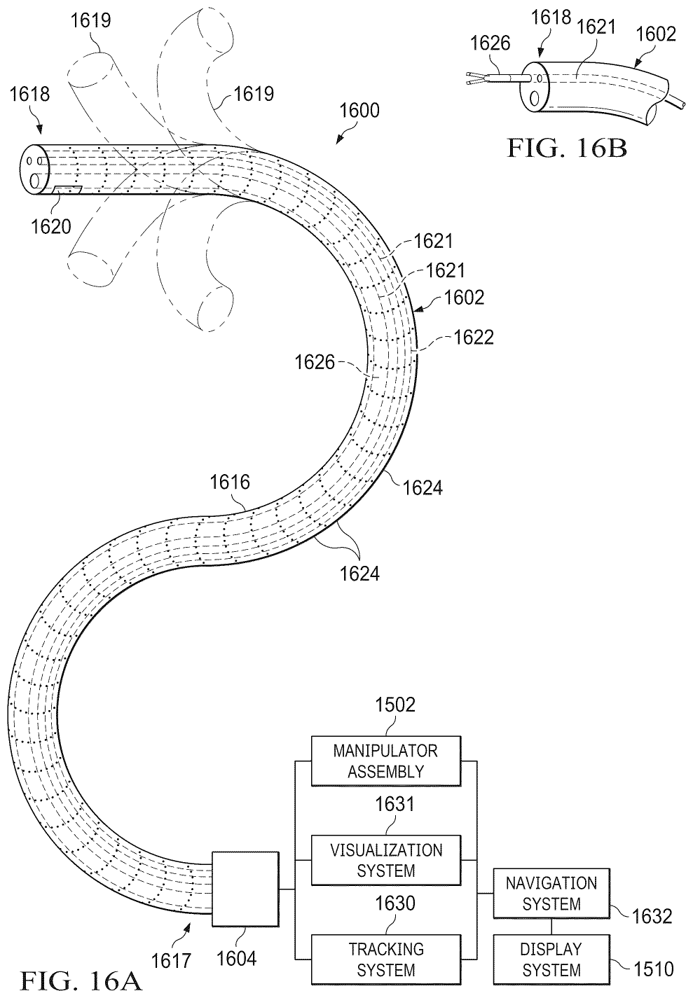

A shape sensor (e.g., the shape sensor 1622 of FIG. 16A) may be positioned within the sensor lumen 290. The sensor lumen 290 may terminate within the distal end 204 of the elongate flexible device 200 such that the sensor lumen 290 does not have a distal opening. The shape sensor extends through the sensor lumen 290 from a proximal end of the elongate flexible device 200 to the distal end 204 of the elongate flexible device 200. Alternatively, the shape sensor may be embedded within a wall of the elongate flexible device 200. A shape of the elongate flexible device 200 may be determined based on data (e.g., shape data) received from the shape sensor. The shape sensor may be an optical fiber shape sensor or one or more electromagnetic sensors. In some examples, the sensor lumen 290 is positioned as close to the longitudinal axis of the elongate flexible device 200 as possible. This may increase the accuracy with which the shape of the elongate flexible device 200 may be determined based on the shape data received from the shape sensor. Additional details regarding the shape sensor are discussed below with respect to FIG. 16A.

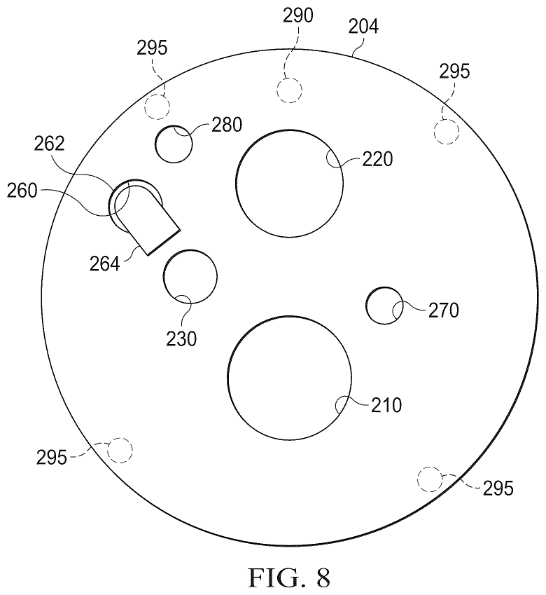

FIG. 8 illustrates a front view of the distal end 204 of the elongate flexible device 200 with additional optional features. For example, the elongate flexible device 200 optionally includes pull wires that are positioned in pull wire lumens 295. Four pull wire lumens 295 are shown in FIG. 8, but the elongate flexible device 200 may include any other number of pull wires and pull wire lumens 295, such as three lumens, five lumens, six lumens, or any other number of lumens. In some examples, the pull wires may be embedded in the wall of the elongate flexible device 200 instead of being positioned within the pull wire lumens 295. The pull wires are used to articulate the elongate flexible device 200. The pull wires may be controlled manually, via robotically-assisted control, or via a combination of manual and robotically-assisted control, as discussed above with respect to FIG. 1.

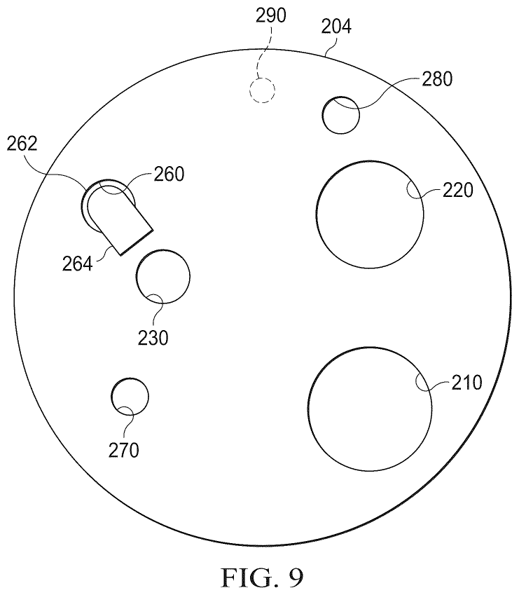

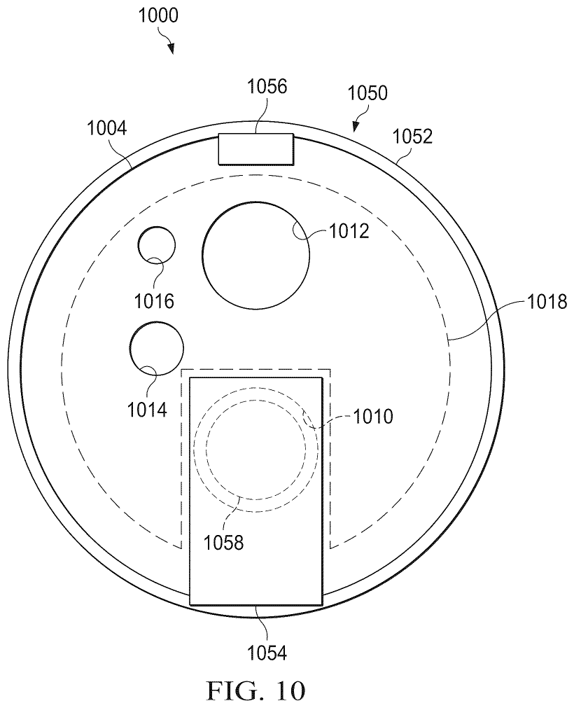

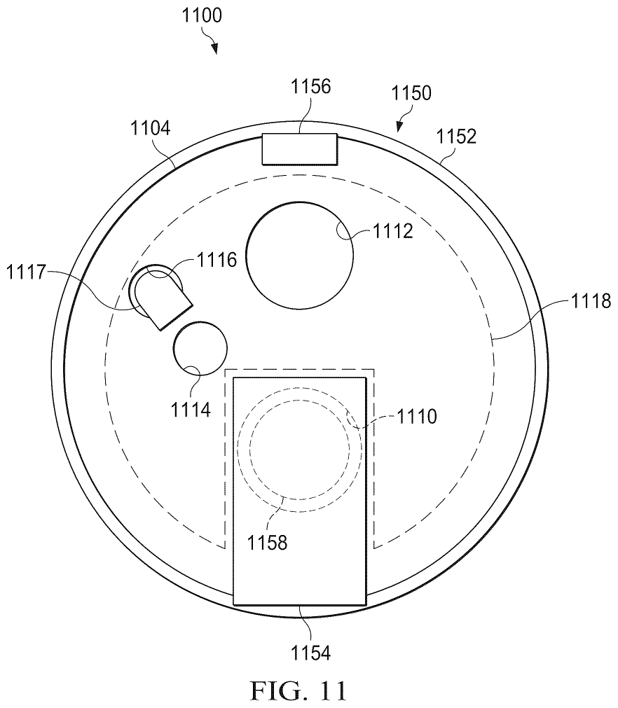

FIG. 9 illustrates a front view of the distal end 204 of the elongate flexible device 200 with various working channels and lumens arranged in an alternative configuration. In FIG. 9, the working channel 210 and the working channel 220 are shown as being positioned closer to a side of the elongate flexible device 200 rather more centrally location in the elongate flexible device 200, as shown in FIG. 8, for example. Additionally, when comparing at least FIGS. 8 and 9, FIG. 9 illustrates the irrigation lumen 270 in a different position than the position of the irrigation lumen 270 shown in FIG. 8. Additionally, FIG. 9 illustrates the illumination lumen 280 in a different position than the position of the illumination lumen 280 shown in FIG. 8. Any of the other lumens may be positioned in different locations in the elongate flexible device 200 than the positions of the lumens currently illustrated in the figures. Some exemplary configurations of the elongate flexible device 200 will be discussed with respect to FIGS. 10 and 11.

FIG. 10 illustrates a front view of a distal end 1004 of an elongate flexible device 1000. The elongate flexible device 1000 may be the elongate flexible device 200, and the discussion above with respect to the elongate flexible device 200 similarly applies and corresponds to the elongate flexible device 1000. The elongate flexible device 1000 includes a working channel 1010 (e.g., the working channel 210), a working channel 1012 (e.g., the working channel 220), an imaging lumen 1014 (e.g., the imaging lumen 230), and an illumination lumen 1016 (e.g., the illumination lumen 280). The working channel 1010, the working channel 1012, the imaging lumen 1014, and the illumination lumen 1016 are all positioned within an operational space 1018 (e.g., the operational space 240).

A tool 1050 (e.g., the tool 300) is coupled to the distal end 1004 of the elongate flexible device 1000. The tool 1050 includes a rim 1052 (e.g., the rim 310) that may surround the distal end 1004 of the elongate flexible device 1000. The tool 1050 also includes an extension member 1054 (e.g., the extension member 320) and an extension member 1056 (e.g., the extension member 330). The extension member 1054 includes an attachment portion 1058 (e.g., the attachment portion 340). In FIG. 10, the attachment portion 1058 is shown as being positioned within the working channel 1012.

In some examples, an illumination device is positioned within the illumination lumen 1016. The illumination device may be a fiber optic illumination device or any other type of illumination device. The illumination device is used to emit light to illuminate some or all of the operational space 1018 and/or some or all of the target anatomy 250. An imaging device in the imaging lumen 1014 is used to capture images of one or more portions of the operational space 1018, any instruments or tools positioned in the operational space 1018, and/or one or more portions of the target anatomy 250. The illumination light provided by the illumination device is used to enhance the clarity of the images captured by the imaging device by providing light to the area being imaged by the imaging device.

In some examples, the illumination light may produce a glare in the image captured by the imaging device depending on the position of the illumination lumen 1016 and the positions of the extension member 1054 and the extension member 1056. For example, if the illumination lumen 1016 is partially obstructed by the extension member 1054 or the extension member 1056, the glare caused by the illumination light may increase. This is due at least in part to the fact that more light will reflect off of the extension member that is obstructing the illumination lumen 1016. Additionally, as the position of the illumination lumen 1016 gets closer to the extension member 1054 or to the extension member 1056, the glare caused by the illumination light will increase as a result of an increased amount of illumination light reflecting off of the extension member 1054 or the extension member 1056.

To reduce the amount of glare caused by the illumination light, the illumination lumen(s) 1016 may be positioned closer to the outer edge of the elongate flexible device 1000 or at least farther away from the extension members 1054, 1056. By placing the illumination lumen(s) 1016 in such positions, more of the target anatomy 250 may be illuminated, and the amount of glare caused by the illumination light may be reduced.

Additionally or alternatively, to reduce the amount of glare caused by the illumination light, only one illumination lumen 1016 may be included in the elongate flexible device 1000. This reduces the overall amount of illumination light that is produced, which may reduce the amount of glare caused by the illumination light. However, by reducing the amount of the illumination light, portions of the images captured by the imaging device that are shadowed may increase. Therefore, the number of illumination lumens and position of those illumination lumens may be adjusted to balance a ratio between the amount of glare caused by the illumination light and the amount of shadows in the images to ultimately ensure that a clear image is captured by the imaging device.

Additionally or alternatively, to reduce the amount of glare caused by the illumination light, the tool 1050 or at least the extension members 1054, 1056 of the tool 1050 may be coated with a glare reduction coating, which may include magnesium fluoride, zinc sulfide, silicon nitride, or any other suitable material or compound used to reduce glare. Additionally or alternatively, to reduce the amount of glare in the images captured by the image device, image processing may be performed on the images captured by the imaging device. For example, one or more processors of a control system (e.g., the one or more processors 1514 of the control system 1512 in FIG. 15) and/or a visualization system (e.g., the visualization system 1631 in FIG. 16A) may perform auto-exposure image processing. This image processing may be used to reduce the amount of glare in the portions of the captured images where the tool 1050 is displayed without darkening the other portions of the images. For example, the amount of glare in the portions of the captured images where the tool 1050 is displayed may be reduced while the brightness of the other portions of the captured images remains unchanged.

FIG. 11 illustrates a front view of a distal end 1104 of an elongate flexible device 1100. The elongate flexible device 1100 may be the elongate flexible device 200, and the discussion above with respect to the elongate flexible device 200 similarly applies and corresponds to the elongate flexible device 1100. The elongate flexible device 1100 includes a working channel 1110 (e.g., the working channel 210), a working channel 1112 (e.g., the working channel 220), an imaging lumen 1114 (e.g., the imaging lumen 230), and a fluid lumen 1116 (e.g., the fluid lumen 260). The working channel 1110, the working channel 1112, the imaging lumen 1114, and the fluid lumen 1116 are all positioned within an operational space 1118 (e.g., the operational space 240).

A tool 1150 (e.g., the tool 300) is coupled to the distal end 1104 of the elongate flexible device 1100. The tool 1150 includes a rim 1152 (e.g., the rim 310) that may surround the distal end 1104 of the elongate flexible device 1100. The tool 1150 also includes an extension member 1154 (e.g., the extension member 320) and an extension member 1156 (e.g., the extension member 330). The extension member 1154 includes an attachment portion 1158 (e.g., the attachment portion 340). In FIG. 11, the attachment portion 1158 is shown as being positioned within the working channel 1112.

A nozzle 1117 may be located at the distal end of the fluid lumen 1116. The nozzle 1117 is used to direct the fluid from the fluid lumen 1116 and onto the imaging device that is positioned within the imaging lumen 1114. This fluid can clean a lens of the imaging device to help the imaging device capture clearer pictures of the target anatomy 250 and the medical instrument(s) that may be positioned in the operational space 1118. In some examples, the working channel 1110 and the imaging lumen 1114 are aligned with the nozzle 1117 such that when fluid is discharged from the nozzle 1117, the fluid is directed over the lens of the imaging device in the imaging lumen 1114 and over the extension member 1154 of the tool 1150. Thus, the discharged fluid may be used to clean both the lens of the imaging device and the extension member 1154. This may allow for more components of the elongate flexible device 1100 and the tool 1150 to be cleaned while reducing the total amount of fluid that is discharged into the patient anatomy.

Additionally or alternatively, while fluid is being discharged from the nozzle 1017 or after fluid has been discharged from the nozzle 1117, the elongate flexible device 1100 may be rotated and/or articulated to allow the fluid stream to clean other features of the elongate flexible device 1100 and/or the tool 1150. In some alternative examples, instead of a nozzle, a fluid dispersion device (e.g., a “shower-head-like” device) may be located at the end of the fluid lumen 1116. The fluid dispersion device may include a plurality of holes that are spaced radially and circumferentially such that when fluid is discharged through the fluid dispersion device, most or all of the distal end 1104 of the elongate flexible device 1100 and the component(s) coupled to it, such as the tool 1150, may be simultaneously cleaned.

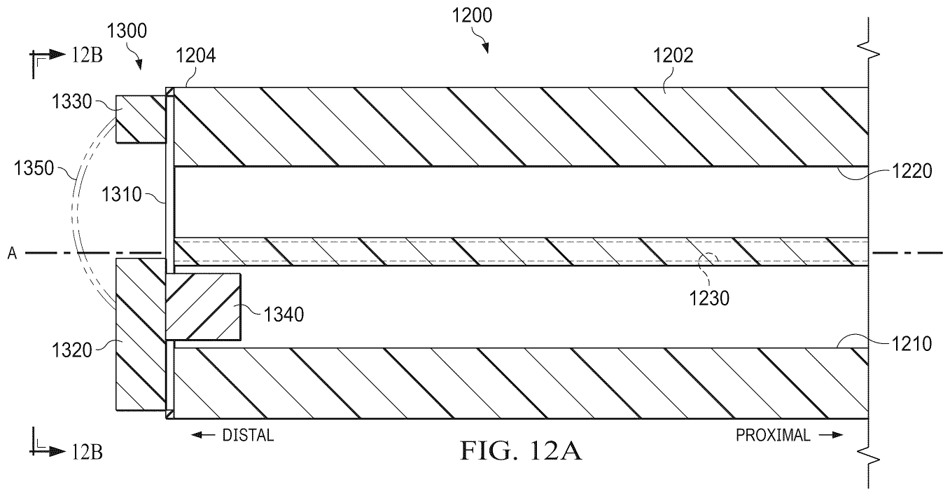

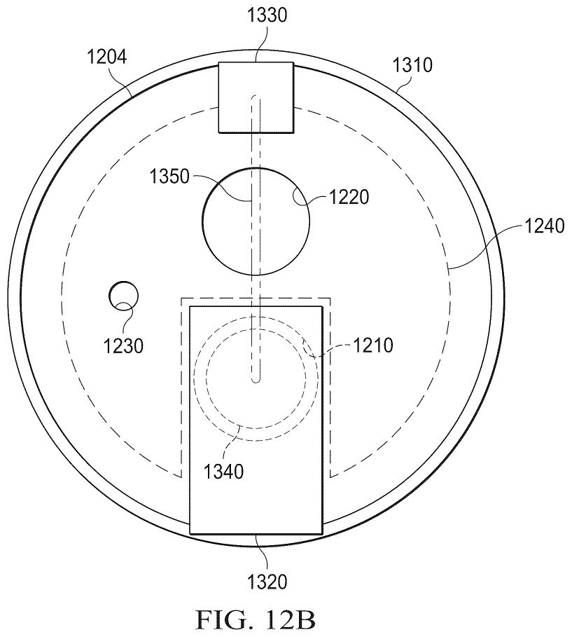

FIG. 12A illustrates a cross-sectional side view of an elongate flexible device 1200. The elongate flexible device 1200 includes a body portion 1202 and the distal end 1204. The elongate flexible device 1200 may be the elongate flexible device 200, and the discussion above with respect to the elongate flexible device 200 similarly applies and corresponds to the elongate flexible device 1200. The elongate flexible device 1200 further includes a working channel 1210 (e.g., the working channel 210), a working channel 1220 (e.g., the working channel 220), and an imaging lumen 1230 (e.g., the imaging lumen 230). A tool 1300 may be coupled to the distal end 1204 of the elongate flexible device 1200. The tool 1300 may be the tool 300, and the discussion above with respect to the tool 300 similarly applies and corresponds to the tool 1300. The tool 1300 includes a rim 1310 (e.g., the rim 310), an extension member 1320 (e.g., the extension member 320), and an extension member 1330 (e.g., the extension member 330). The extension member 1320 may include an attachment portion 1340 (e.g., the attachment portion 340). As shown in FIG. 12A, when the tool 1300 is coupled to the elongate flexible device 1200, the attachment portion 1340 extends into the working channel 1210. The attachment portion 1340 may include one or more expandable members (not shown), such as one or more balloons, one or more springs, or any other expandable member, to assist with coupling the tool 1300 to the elongate flexible device 1200.

In some examples, the tool 1300 is a suturing tool. The suturing tool includes a needle that is used to suture one or more portions of a target anatomy (e.g., the target anatomy 250). The tool 1300 includes a tool path 1350 defined between the extension member 1320 and the extension member 1330. The tool path 1350 may be a needle path. The needle path 1350 defines the path a needle travels between the extension members 1320, 1330. The needle is used to apply sutures to the target anatomy. The needle may travel from the extension member 1320 to the extension member 1330. Or the needle may travel from the extension member 1330 to the extension member 1320. Additionally, or alternatively, the needle may continuously travel between the extension member 1320 and the extension member 1330 until the procedure is complete. In FIG. 12A, the needle path 1350 is depicted as a curved path. However, in other examples, the needle path 1350 may be a straight line, such as a straight line that is transverse to a longitudinal axis A of the elongate flexible device 1200. The needle path 1350 may take a path of any other shape that travels between the extension members 1320, 1330.

FIG. 12B illustrates a front view of the tool 1300 coupled to the distal end 1204 of the elongate flexible device 1200. The working channel 1220 is aligned with the needle path 1350. In some examples, a longitudinal axis of the working channel 1220 intersects the needle path 1350. Optionally, the working channel 1220 is positioned halfway between the working channel 1210 and the extension member 1330. In other examples, the needle path 1350 overlaps at least a portion of the working channel 1220. In some examples, an interventional tool (not shown) positioned within the working channel 1220 may extend out of the working channel 1220 and acquire a portion of tissue from the target anatomy. The interventional tool may then be used to pull the tissue back towards the distal end 1204 of the elongate flexible device 1200 such that the acquired tissue overlaps the needle path 1350. Therefore, when the needle travels from one extension member, such as the extension member 1330, to another extension member, such as the extension member 1320, the needle travels through the portion of the target anatomy at a certain bite depth. When the working channel 1220 is aligned with the needle path 1350, the interventional tool may more easily bring the acquired tissue into the needle path 1350 during the suturing procedure. This may also allow for the needle to achieve a maximum tissue bite depth.

When the longitudinal axis of the working channel 1220 intersects the needle path 1350, the tool may be able to acquire the tissue and pull it toward the distal end 1204 to allow the needle to achieve a desired bite depth into the target anatomy for a particular suturing procedure. Each suturing procedure may have a different desired bite depth. The available bite depth may depend on the type of suturing tool being used, the type of target anatomy, the location of one or more critical structures that may be in close proximity to the target anatomy, the location of the target anatomy in the patient anatomy, etc. By aligning the longitudinal axis of the working channel 1220 with the needle path 1350, the suturing procedure may be able to be performed more efficiently and more effectively.

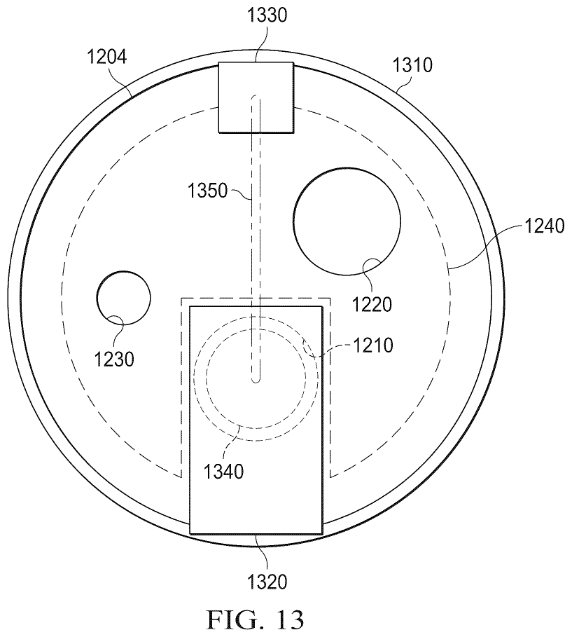

FIG. 13 illustrates a front view of the tool 1300 coupled to the distal end 1204 of the elongate flexible device 1200. In FIG. 13, the longitudinal axis of the working channel 1220 does not intersect the needle path 1350. In fact, the entire working channel 1220 is misaligned with the needle path 1350. In this example, after the interventional tool extends out of the working channel 1220 and acquires tissue (e.g., grasps tissue), the elongate flexible device 1200 may be articulated to bring the tissue into the needle path 1350. Therefore, even if the working channel 1220 is not aligned with the needle path 1350, the suturing tool 1300 can still suture the target anatomy 250 with the needle travelling to a desired bite depth. The working channel 1220 may be sized such that if the needle of the suturing tool is activated while the interventional tool is extended distally from the working channel 1220, the interventional tool is pushed off to the side out of the needle path 1350 rather than being pierced by the needle. For example, a diameter of the working channel 1220 may be larger than an outer diameter of the interventional tool.

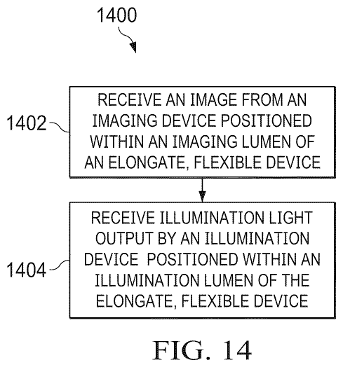

FIG. 14 is a flowchart illustrating a method 1400 of visualizing the target anatomy 250 based on images received from an imaging device in an elongate flexible device (e.g., the elongate flexible device 200). The method 1400 is illustrated as a set of operations or processes 1402 through 1404. The processes illustrated in FIG. 14 may be performed in a different order than the order shown in FIG. 14, and one or more of the illustrated processes might not be performed in some examples of the method 1400. Additionally, one or more processes that are not expressly illustrated in FIG. 14 may be included before, after, in between, or as part of the illustrated processes.

At a process 1402, an image is received from an imaging device positioned within an imaging lumen (e.g., the imaging lumen 230) of the elongate flexible device 200. The image may be received by an imaging system 1509 (FIG. 15), which may include and/or be in communication with one or more processors. For example, the imaging system 1509 may be in communication with and communicatively coupled to the processor 1514 of the control system 1512. In some examples, the imaging device is a stereoscopic camera. In other examples, the imaging device may be any other type of camera or other image capture device.

At a process 1404, illumination light is received from an illumination device positioned within an illumination lumen (e.g., the illumination lumen 280) of the elongate flexible device 200. The illumination light may be received by the imaging system 1509. In some examples, the illumination device is a fiber optic illumination device. In other examples, the illumination device is any other type of illumination device. In some examples, as discussed above, the elongate flexible device 200 includes more than one illumination lumen 280. A separate illumination device may be positioned within each, respective illumination lumen 280 such that multiple illumination devices are used to provide illumination from the distal end 204 of the elongate flexible device 200.

As discussed above with respect to FIG. 10, in some examples, the illumination light reflects off of an extension member (e.g., the extension member 320 or the extension member 330) of a tool (e.g., the tool 300) coupled to the elongate flexible device 200. In some examples, the illumination light reflects off of both the extension member 320 and the extension member 330. The reflection of the illumination off of the extension member 320 and/or the extension member 330 may create a glare in the image captured by the imaging device.

To reduce the glare, the extension member 320 and/or the extension member 330 are positioned outside of the operational space 240. The operational space 240 extends between the target anatomy 250 and a distal end of the imaging lumen 230 and a distal end of the illumination lumen 280. Additionally or alternatively, the illumination lumen 280 may be spaced from the extension member 320 and/or the extension member 330 to reduce the glare in the image captured by the imaging device. Additionally or alternatively, the extension member 320 and/or the extension member 330 may be coated with a glare reduction coating to reduce the glare in the image captured by the imaging device.

In some examples, the components discussed above may be used in a procedure performed with a teleoperated system as described in further detail below. The teleoperated system may be suitable for use in, for example, medical, teleoperated medical, surgical, diagnostic, therapeutic, or biopsy procedures. While some examples are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems and general robotic, general teleoperational, or robotic medical systems.

FIG. 15 illustrates an embodiment of a robotically-assisted manipulator system for use with the tools described herein. The manipulator system can be used, for example, in surgical, diagnostic, therapeutic, biopsy, or non-medical procedures, and is generally indicated by the reference numeral 1500. As shown in FIG. 15, a robotically-assisted manipulator system 1500 can include one or more manipulator assemblies 1502 for operating one or more medical instrument systems 1504 in performing various procedures on a patient P positioned on a table T in a medical environment 1501. For example, the manipulator assembly 1502 can drive catheter or end effector motion, can apply treatment to target tissue, and/or can manipulate control members. The manipulator assembly 1502 can be teleoperated, non-teleoperated, or a hybrid teleoperated and non-teleoperated assembly with select degrees of freedom of motion that can be motorized and/or teleoperated and select degrees of freedom of motion that can be non-motorized and/or non-teleoperated. An operator input system 1506, which can be inside or outside of the medical environment 1501, generally includes one or more control devices for controlling manipulator assembly 1502. Manipulator assembly 1502 supports medical instrument system 1504 and can optionally include a plurality of actuators or motors that drive inputs on medical instrument system 1504 in response to commands from a control system 1512. The actuators can optionally include drive systems that when coupled to medical instrument system 1504 can advance medical instrument system 1504 into a naturally or surgically created anatomic orifice. Other drive systems can move the distal end of medical instrument in multiple degrees of freedom, which can include three degrees of linear motion (e.g., linear motion along the X, Y, Z Cartesian axes) and in three degrees of rotational motion (e.g., rotation about the X, Y, Z Cartesian axes). The manipulator assembly 1502 can support various other systems for irrigation, treatment, or other purposes. Such systems can include fluid systems (including, for example, reservoirs, heating/cooling elements, pumps, and valves), generators, lasers, interrogators, and ablation components.

Robotically-assisted manipulator system 1500 also includes a display system 1510 for displaying an image or representation of the surgical site and medical instrument system 1504 generated by an imaging system 1509 which can include an imaging system, such as an endoscopic imaging system. Display system 1510 and operator input system 1506 can be oriented so an operator O can control medical instrument system 1504 and operator input system 1506 with the perception of telepresence. A graphical user interface can be displayable on the display system 1510 and/or a display system of an independent planning workstation.

In some examples, the endoscopic imaging system components of the imaging system 1509 can be integrally or removably coupled to medical instrument system 1504. However, in some examples, a separate imaging device, such as an endoscope, attached to a separate manipulator assembly can be used with medical instrument system 1504 to image the surgical site. The endoscopic imaging system 1509 can be implemented as hardware, firmware, software, or a combination thereof which interact with or are otherwise executed by one or more computer processors, which can include the processors of the control system 1512.

Robotically-assisted manipulator system 1500 can also include a sensor system 1508. The sensor system 1508 can include a position/location sensor system (e.g., an actuator encoder or an electromagnetic (EM) sensor system) and/or a shape sensor system (e.g., an optical fiber shape sensor) for determining the position, orientation, speed, velocity, pose, and/or shape of the medical instrument system 1504. The sensor system 1508 can also include temperature, pressure, force, or contact sensors or the like.

Robotically-assisted manipulator system 1500 can also include a control system 1512. Control system 1512 includes at least one memory 1516 and at least one computer processor 1514 for effecting control between medical instrument system 1504, operator input system 1506, sensor system 1508, and display system 1510. Control system 1512 also includes programmed instructions (e.g., a non-transitory machine-readable medium storing the instructions) to implement a procedure using the robotically-assisted manipulator system including for navigation, steering, imaging, engagement feature deployment or retraction, applying treatment to target tissue (e.g., via the application of energy), or the like.

Control system 1512 can optionally further include a virtual visualization system to provide navigation assistance to operator O when controlling medical instrument system 1504 during an image-guided surgical procedure. Virtual navigation using the virtual visualization system can be based upon reference to an acquired pre-operative or intra-operative dataset of anatomic passageways. The virtual visualization system processes images of the surgical site imaged using imaging technology such as computerized tomography (CT), magnetic resonance imaging (MRI), fluoroscopy, thermography, ultrasound, optical coherence tomography (OCT), thermal imaging, impedance imaging, laser imaging, nanotube X-ray imaging, and/or the like. The control system 1512 can use a pre-operative image to locate the target tissue (using vision imaging techniques and/or by receiving user input) and create a pre-operative plan, including an optimal first location for performing treatment. The pre-operative plan can include, for example, a planned size to expand an expandable device, a treatment duration, a treatment temperature, and/or multiple deployment locations.

FIG. 16A shows a medical instrument system 1600 according to some embodiments. In some embodiments, medical instrument system 1600 can be used in an image-guided medical procedure. In some examples, medical instrument system 1600 can be used for non-teleoperational exploratory procedures or in procedures involving traditional manually operated medical instruments, such as endoscopy. In some embodiments, medical instrument system 1600 is interchangeable with, or a variation of, medical instrument system 1504 of FIG. 15.

Medical instrument system 1600 includes elongate flexible device 1602, such as a flexible catheter or endoscope (e.g., gastroscope, bronchoscope), coupled to a drive unit 1604. Elongate flexible device 1602 includes a flexible body 1616 having proximal end 1617 and distal end, or tip portion, 1618. In some embodiments, flexible body 1616 has an approximately 14-20 mm outer diameter. Other flexible body outer diameters can be larger or smaller. Flexible body 1616 can have an appropriate length to reach certain portions of the anatomy, such as the lungs, sinuses, throat, or the upper or lower gastrointestinal region, when flexible body 1616 is inserted into a patient's oral or nasal cavity.

Medical instrument system 1600 optionally includes a tracking system 1630 for determining the position, orientation, speed, velocity, pose, and/or shape of distal end 1618 and/or of one or more segments 1624 along flexible body 1616 using one or more sensors and/or imaging devices. The entire length of flexible body 1616, between distal end 1618 and proximal end 1617, can be effectively divided into segments 1624. Tracking system 1630 can optionally be implemented as hardware, firmware, software or a combination thereof which interact with or are otherwise executed by one or more computer processors, which can include the processors of control system 1512 in FIG. 15.

Tracking system 1630 can optionally track distal end 1618 and/or one or more of the segments 1624 using a shape sensor 1622. In some embodiments, tracking system 1630 can optionally and/or additionally track distal end 1618 using a position sensor system 1620, such as an electromagnetic (EM) sensor system. In some examples, position sensor system 1620 can be configured and positioned to measure six degrees of freedom, e.g., three position coordinates X, Y, Z and three orientation angles indicating pitch, yaw, and roll of a base point or five degrees of freedom, e.g., three position coordinates X, Y, Z and two orientation angles indicating pitch and yaw of a base point.

Flexible body 1616 includes one or more channels 1621 (e.g., any of the working channels or lumens discussed above) sized and shaped to receive one or more medical instruments 1626. In some embodiments, flexible body 1616 includes two channels 1621 for separate instruments 1626, however, a different number of channels 1621 can be provided. FIG. 16B is a simplified diagram of flexible body 1616 with medical instrument 1626 extended according to some embodiments. In some embodiments, medical instrument 1626 can be used for procedures and aspects of procedures, such as surgery, biopsy, ablation, mapping, imaging, illumination, irrigation, or suction. Medical instrument 1626 can be deployed through channel 1621 of flexible body 1616 and used at a target location within the anatomy. Medical instrument 1626 can include, for example, image capture devices, biopsy instruments, ablation instruments, catheters, laser ablation fibers, and/or other surgical, diagnostic, or therapeutic tools. Medical tools can include end effectors having a single working member such as a scalpel, a blunt blade, a lens, an optical fiber, an electrode, and/or the like. Other end effectors can include, for example, forceps, graspers, balloons, needles, scissors, clip appliers, and/or the like. Other end effectors can further include electrically activated end effectors such as electrosurgical electrodes, transducers, sensors, imaging devices and/or the like. Medical instrument 1626 can be advanced from the opening of channel 1621 to perform the procedure and then retracted back into the channel when the procedure is complete. Medical instrument 1626 can be removed from proximal end 1617 of flexible body 1616 or from another optional instrument port (not shown) along flexible body 216. The medical instrument 1626 can be used with an image capture device (e.g., an endoscopic camera) also within the elongate flexible device 1602. Alternatively, the medical instrument 1626 can itself be the image capture device.

Medical instrument 1626 can additionally house cables, linkages, or other actuation controls (not shown) that extend between its proximal and distal ends to controllably the bend distal end of medical instrument 1626. Flexible body 1616 can also house cables, linkages, or other steering controls (not shown) that extend between drive unit 1604 and distal end 1618 to controllably bend distal end 1618 as shown, for example, by broken dashed line depictions 1619 of distal end 1618. In some examples, at least four cables are used to provide independent “up-down” steering to control a pitch motion of distal end 1618 and “left-right” steering to control a yaw motion of distal end 1618. In embodiments in which medical instrument system 1600 is actuated by a robotically-assisted assembly, drive unit 1604 can include drive inputs that removably couple to and receive power from drive elements, such as actuators, of the teleoperational assembly. In some embodiments, medical instrument system 1600 can include gripping features, manual actuators, or other components for manually controlling the motion of medical instrument system 1600. The information from tracking system 1630 can be sent to a navigation system 1632 where it is combined with information from visualization system 1631 and/or the preoperatively obtained models to provide the physician or other operator with real-time position information.

In addition, the singular forms “a”, “an”, and “the” are intended to include the plural forms as well, unless the context indicates otherwise. And the terms “comprises,” “comprising,” “includes,” “has,” and the like specify the presence of stated features, steps, operations, elements, and/or components but do not preclude the presence or addition of one or more other features, steps, operations, elements, components, and/or groups. Components described as coupled may be electrically or mechanically directly coupled, or they may be indirectly coupled via one or more intermediate components. The auxiliary verb “may” likewise implies that a feature, step, operation, element, or component is optional.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

A computer is a machine that follows programmed instructions to perform mathematical or logical functions on input information to produce processed output information. A computer includes a logic unit that performs the mathematical or logical functions, and memory that stores the programmed instructions, the input information, and the output information. The term “computer” and similar terms, such as “processor” or “controller” or “control system”, are analogous.

Although some of the examples described herein refer to surgical procedures or instruments, or medical procedures and medical instruments, the techniques disclosed apply to non-medical procedures and non-medical instruments. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy), and performing procedures on human or animal cadavers. Further, these techniques can also be used for surgical and nonsurgical medical treatment or diagnosis procedures.

Further, although some of the examples presented in this disclosure discuss teleoperational robotic systems or remotely operable systems, the techniques disclosed are also applicable to computer-assisted systems that are directly and manually moved by operators, in part or in whole.

Additionally, one or more elements in examples of this disclosure may be implemented in software to execute on a processor of a computer system such as a control processing system. When implemented in software, the elements of the examples of the present disclosure are essentially the code segments to perform the necessary tasks. The program or code segments can be stored in a processor readable storage medium or device that may have been downloaded by way of a computer data signal embodied in a carrier wave over a transmission medium or a communication link. The processor readable storage device may include any medium that can store information including an optical medium, semiconductor medium, and magnetic medium. Processor readable storage device examples include an electronic circuit, a semiconductor device, a semiconductor memory device, a read only memory (ROM), a flash memory, an crasable programmable read only memory (EPROM); a floppy diskette, a CD-ROM, an optical disk, a hard disk, or other storage device. The code segments may be downloaded via computer networks such as the Internet, Intranet, etc.

Note that the processes and displays presented may not inherently be related to any particular computer or other apparatus, and various systems may be used with programs in accordance with the teachings herein. The required structure for a variety of the systems discussed above will appear as elements in the claims. In addition, the examples of the present disclosure are not described with reference to any particular programming language. It will be appreciated that a variety of programming languages may be used to implement the teachings of the present disclosure as described herein.

While certain examples of the present disclosure have been described and shown in the accompanying drawings, it is to be understood that such examples are merely illustrative of and not restrictive to the broad disclosed concepts, and that the examples of the present disclosure not be limited to the specific constructions and arrangements shown and described, since various other modifications may occur to those ordinarily skilled in the art.

1. A system, comprising:
an elongate flexible device, comprising:
a first working channel including a distal end;
a second working channel including a distal end; and
an imaging lumen including a distal end, the imaging lumen configured to receive an imaging device,
wherein an operational space extends between a target anatomy and the distal end of the first working channel, the distal end of the second working channel, and the distal end of the imaging lumen; and
a tool removably couplable to a distal end of the elongate flexible device, the tool comprising:
a first extension member configured to extend distally from the distal end of the elongate flexible device; and
a second extension member configured to extend distally from the distal end of the elongate flexible device,
wherein when the tool is coupled to the distal end of the elongate flexible device, the first extension member and the second extension member are positioned outside of the operational space such that the operational space is free from obstruction by the first extension member and the second extension member.

2. The system of claim 1, wherein when the tool is coupled to the distal end of the elongate flexible device, an attachment portion of the first extension member extends within the first working channel of the elongate flexible device.

3. The system of claim 2, wherein the attachment portion includes a locking feature that rotationally locks the tool relative to the elongate flexible device.

4. The system of claim 1, wherein the elongate flexible device further comprises:
an illumination lumen including a distal end, the illumination lumen configured to receive an illumination device;
an irrigation lumen including a distal end; and
a fluid lumen including a distal end.

5. The system of claim 4, wherein a nozzle is positioned at the distal end of the fluid lumen, the nozzle configured to direct fluid onto the imaging device in the imaging lumen.

6. The system of claim 4, wherein the operational space further extends between the target anatomy and the distal end of the illumination lumen, the distal end of the irrigation lumen, and the distal end of the fluid lumen.

7. The system of claim 1, wherein a tool path extends between the first extension member and the second extension member, and wherein when the tool is coupled to the elongate flexible device, a longitudinal axis of the second working channel intersects the tool path.

8. The system of claim 7, wherein the tool is a suturing tool, the suturing tool including a needle, and wherein the tool path is a needle path of the needle.

9. The system of claim 1, wherein a tool path extends between the first extension member and the second extension member, and wherein when the tool is coupled to the elongate flexible device, the second working channel is misaligned with the tool path.

10. The system of claim 1, wherein the elongate flexible device further comprises one or more pull wires, and wherein the elongate flexible device is steerable via actuation of the one or more pull wires.

11. The system of claim 10, wherein the elongate flexible device further comprises a plurality of pull wire lumens, wherein each pull wire lumen is configured to receive a respective pull wire of the one or more pull wires.

12. The system of claim 10, wherein the one or more pull wires are embedded in a wall of the elongate flexible device.

13. A system, comprising:
an elongate flexible device, comprising:
a first working channel including a distal end;
a second working channel including a distal end; and
an imaging lumen including a distal end, the imaging lumen configured to receive an imaging device,
wherein an operational space extends between a target anatomy and the distal end of the first working channel, the distal end of the second working channel, and the distal end of the imaging lumen,
wherein when a tool is coupled to a distal end of the elongate flexible device, a first extension member of the tool and a second extension member of the tool are positioned outside of the operational space such that the operational space is free from obstruction by the first extension member and the second extension member, wherein the first extension member is configured to extend distally from the distal end of the elongate flexible device, and wherein the second extension member configured to extend distally from the distal end of the elongate flexible device.

14. The system of claim 13, wherein when the tool is coupled to the distal end of the elongate flexible device, an attachment portion of the first extension member extends within the first working channel of the elongate flexible device.

15. The system of claim 14, wherein the attachment portion includes a locking feature that rotationally locks the tool relative to the elongate flexible device.

16. The system of claim 13, wherein the elongate flexible device further comprises:
an illumination lumen including a distal end, the illumination lumen configured to receive an illumination device;
an irrigation lumen including a distal end; and
a fluid lumen including a distal end.

17. The system of claim 16, wherein a nozzle is positioned at the distal end of the fluid lumen, the nozzle configured to direct fluid onto the imaging device in the imaging lumen.

18. The system of claim 16, wherein the operational space further extends between the target anatomy and the distal end of the illumination lumen, the distal end of the irrigation lumen, and the distal end of the fluid lumen.

19. The system of claim 13, wherein a tool path extends between the first extension member and the second extension member, and wherein when the tool is coupled to the elongate flexible device, a longitudinal axis of the second working channel intersects the tool path.

20. The system of claim 19, wherein the tool is a suturing tool, the suturing tool including a needle, and wherein the tool path is a needle path of the needle.

21. The system of claim 13, wherein a tool path extends between the first extension member and the second extension member, and wherein when the tool is coupled to the elongate flexible device, the second working channel is misaligned with the tool path.

22. The system of claim 13, wherein the elongate flexible device further comprises one or more pull wires, and wherein the elongate flexible device is steerable via actuation of the one or more pull wires.

23-44. (canceled)
